(19) United States
(12) Patent Application Publication
(10) Pub. No.: US 2023/0143477 A1
(43) Pub. Date: May 11, 2023

(54) Subcutaneous Port With Locking Member

(71) Applicant: Takeda Pharmaceutical Company Limited, Osaka (JP)

(72) Inventors: Ayesha Arzumand, Lexington, MA (US); Chris Tarapata, North Andover, MA (US); Marc Bellisario, Tewksbury, MA (US); Christel Hartshorn, Nashua, NH (US); Matthew J. Gunn, North Wales, PA (US); Chase Fetzer, Belmont, MA (US)

(73) Assignee: Takeda Pharmaceutical Company Limited, Osaka (JP)

(21) Appl. No.: 17/995,198

(22) PCT Filed: Apr. 2, 2021
(86) PCT No.: PCT/JP2021/014310

§ 371 Date: Sep. 30, 2022

Related U.S. Application Data
(60) Provisional application No. 63/004,142, filed on Apr. 2, 2020.

(51) Int. Cl. A61M 39/02; A61M 39/04

(52) U.S. Cl. CPC A61M 39/0208; A61M 39/04

(57) ABSTRACT

A subcutaneous port assembly includes a base, a connector, a stem, a sealing element, and a locking member. The connector extends from a first end attached to the base to a distal end and includes an inner surface defining a socket having an inside diameter. The stem extends from the base and into the socket. The stem includes an outer surface having an outside diameter that is less than the inside diameter of the socket. The sealing element is disposed within the socket between the stem and the inner surface of the socket. The locking member has a plunger received within the socket from the distal end and having a terminal end facing the sealing element. The plunger is axially movable between a first position and a second position to selectively compress the sealing element within the socket.

### CROSS REFERENCE TO RELATED APPLICATIONS

This application claims priority under 35 U.S.C. § 119(e) to U.S. Provisional Application 63/004,142 filed on Apr. 2, 2020. The disclosures of this prior application is considered part of the disclosure of this application and is hereby incorporated by reference in its entireties.

### FIELD

The present disclosure relates generally to subcutaneous ports.

### BACKGROUND

This section provides background information related to the present disclosure which is not necessarily prior art.

When connecting a catheter to a subcutaneous port, it may be difficult to securely connect the catheter to the subcutaneous port due to a variety of potential issues, such as anatomical interference(s), surgical equipment interference(s), etc. Additionally, ensuring that the catheter remains connected to the subcutaneous port is important to ensure proper delivery of medication to the patient. Accordingly, there may be room for improvement in the manner in which catheters are connected to subcutaneous ports.

### SUMMARY

This section provides a general summary of the disclosure, and is not a comprehensive disclosure of its full scope or all of its features.

One aspect of the disclosure provides a subcutaneous port assembly comprising a base, a connector, a stem, a sealing element, and a locking member. The connector extends from a first end attached to the base to a distal end, and includes an inner surface defining a socket having an inside diameter. The stem extends from the base and into the socket. The stem includes an outer surface having an outside diameter that is less than the inside diameter of the socket. The sealing element is disposed within the socket between the stem and the inner surface of the socket. The locking member has a plunger received within the socket from the distal end and having a terminal end facing the sealing element. The plunger is axially movable between a first position and a second position to selectively compress the sealing element within the socket.

Implementations of the disclosure may include one or more of the following optional features. In some implementations, the stem is surrounded by the sealing element. Optionally, the sealing element may be spaced apart from the outside diameter of the outer surface of the stem by a first distance in an uncompressed state. In some examples, the plunger in the second position may be configured to axially and radially compress the sealing element towards the outside diameter of the outer surface of the stem.

The locking member may include a cap attached to the plunger. In some examples, the cap includes a position indicator. The position indicator may be configured to communicate a rotational position of the cap. In some example, the cap includes a gripping member and the position indicator is provided on the gripping member. The cap may include a plurality of first threads and the connector may include a plurality of second threads engaged with the first threads. The plunger may move between the first position and the second position via the first threads engaging with the second threads.

The socket may include a first radial protrusion on the inner surface and the plunger may include a second radial protrusion on an outer surface of the plunger, the first radial protrusion and the second radial protrusion configured to selectively engage one another to retain at least a portion of the plunger within the socket.

The sealing element may include a tapered end facing the terminal end of the plunger. The terminal end of the plunger may be configured to axially and radially compress the tapered end of the sealing element when the plunger moves from the first position to the second position.

Another aspect of the disclosure provides a subcutaneous port assembly comprising a base, a connector, a stem, a collar, and a sealing element. The connector extends from a first end adjacent to the base to a second end. The connector includes a neck portion having a first width at the first end and a shoulder portion having a second width closer to the second end, and a socket defined by an inner surface extending through the connector from the second end. The stem extends from the base into the socket, the stem including an outer surface spaced inwardly from the inner surface of the socket. The collar includes an inner biasing surface having a third width that is greater than the first width of the neck portion and less than the second width of the shoulder portion. The collar is operable between a first position where the inner biasing surface is adjacent to the neck portion and a second position where the inner biasing surface is adjacent to the shoulder portion. The sealing element is disposed within the socket between the stem and the inner surface of the socket. The sealing element surrounds the stem when the collar is in the first position and compressed against the stem when the collar is in the second position. This aspect may include one or more of the following optional features.

In some implementations, the stem is surrounded by the connector. Optionally, the collar in the second position may be configured to radially compress the sealing element to secure a catheter between the sealing element and the stem.

The connector may include at least one slot extending from the socket through the shoulder portion. When the collar moves from the first position to the second position, the connector may radially deflect toward the sealing element via the at least one slot. The inner biasing surface may be tapered and the connector may include a tapered outer biasing surface. When the collar moves from the first position to the second position, engagement of the inner biasing surface and the tapered outer surface may cause the connector to radially deflect toward the sealing element and the sealing element to radially compress and secure a catheter to the stem. The tapered outer surface of the connector may be disposed between the neck portion and the shoulder portion. The at least one slot may include a plurality of slots defining a plurality of flexible tabs of the connector. Each flexible tab of the connector may be spaced from an adjacent flexible tab of the connector by one of the slots.

Another aspect of the disclosure provides a locking mechanism for a subcutaneous port assembly, the locking mechanism comprising a connector, a stem, a sealing element, a braided catheter, and a locking member. The connector has a socket and a plurality of threads at a first end of the socket. The stem is disposed within the socket. The sealing element is disposed between the stem and the connector within the socket. The braided catheter has an end coupled to the stem and surrounded by the sealing element. The locking member is slidably coupled to the connector and moveable between a first position and a second position along the connector to selectively change the sealing element between a compressed state and an uncompressed state around the catheter. This aspect may include one or more of the following optional features.

In some implementations, the locking member includes a plunger received within the socket and a cap coupled to the connector by threads. In some examples, the cap includes a position indicator. The position indicator may be configured to communicate a rotational position of the cap. In some example, the cap includes a gripping member and the position indicator is provided on the gripping member. The cap may be movable between a locked position and an unlocked position relative to the connector via engagement of the threads. The connector may include a first radial protrusion on an inner surface and the locking member may include a second radial protrusion on an outer surface. The first radial protrusion and the second radial protrusion may be configured to engage one another to secure at least a portion of the plunger to the connector. The sealing element may include a sleeve having a tapered first end and the plunger may include a tapered second end configured to engage the tapered first end of the sleeve. The tapered first end and the tapered second end may engage when the locking member moves from the first position to the second position, causing the sleeve to axially and radially compress and secure the braided catheter to the stem.

The details of one or more implementations of the disclosure are set forth in the accompanying drawings and the description below. Other aspects, features, and advantages will be apparent from the description and drawings, and from the claims.

### BRIEF DESCRIPTION OF DRAWINGS

The drawings described herein are for illustrative purposes only of selected configurations and are not intended to limit the scope of the present disclosure.

FIG. 1 is a partially-exploded perspective view of a subcutaneous port in accordance with the principles of the present disclosure;

FIG. 2 is an exploded perspective view of the subcutaneous port of FIG. 1;

FIG. 3 is a cross-sectional view of the subcutaneous port of FIG. 1 in an unlocked position taken along the line 3-3;

FIG. 4 is a cross-sectional view of the subcutaneous port of FIG. 1 in a locked position taken along the line 3-3;

FIG. 5A is a cross-sectional detailed view of a portion of the subcutaneous port of FIG. 1 in an unlocked position taken along the line 3-3;

FIG. 5B is a cross-sectional detailed view of a portion of the subcutaneous port of FIG. 1 in an unlocked position and having a flat engagement between a sealing element and a locking member along the line 3-3;

FIG. 6 is a perspective view of another subcutaneous port in accordance with the principles of the present disclosure;

FIG. 7 is a perspective view of the subcutaneous port of FIG. 6 in a locked position;

FIG. 8 is a cross-sectional perspective view of the subcutaneous port of FIG. 6 in an unlocked position taken along line 8-8;

FIG. 9 is a cross-sectional detailed view of the subcutaneous port of FIG. 6 in an unlocked position taken along line 8-8; and

FIG. 10 is a cross-sectional detailed view of the subcutaneous port of FIG. 6 in a locked position taken along line 8-8.

Corresponding reference numerals indicate corresponding parts throughout the drawings.

### DETAILED DESCRIPTION

Example configurations will now be described more fully with reference to the accompanying drawings. Example configurations are provided so that this disclosure will be thorough, and will fully convey the scope of the disclosure to those of ordinary skill in the art. Specific details are set forth such as examples of specific components, devices, and methods, to provide a thorough understanding of configurations of the present disclosure. It will be apparent to those of ordinary skill in the art that specific details need not be employed, that example configurations may be embodied in many different forms, and that the specific details and the example configurations should not be construed to limit the scope of the disclosure.

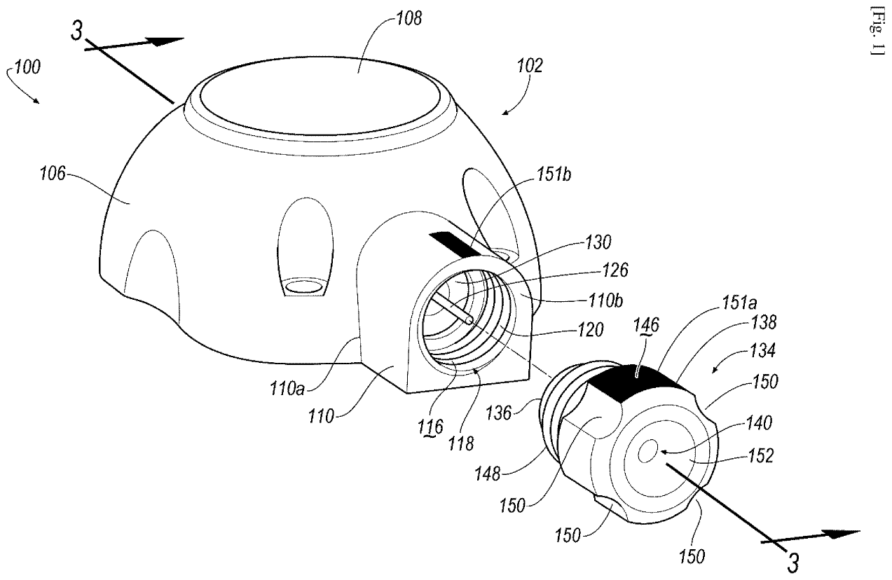

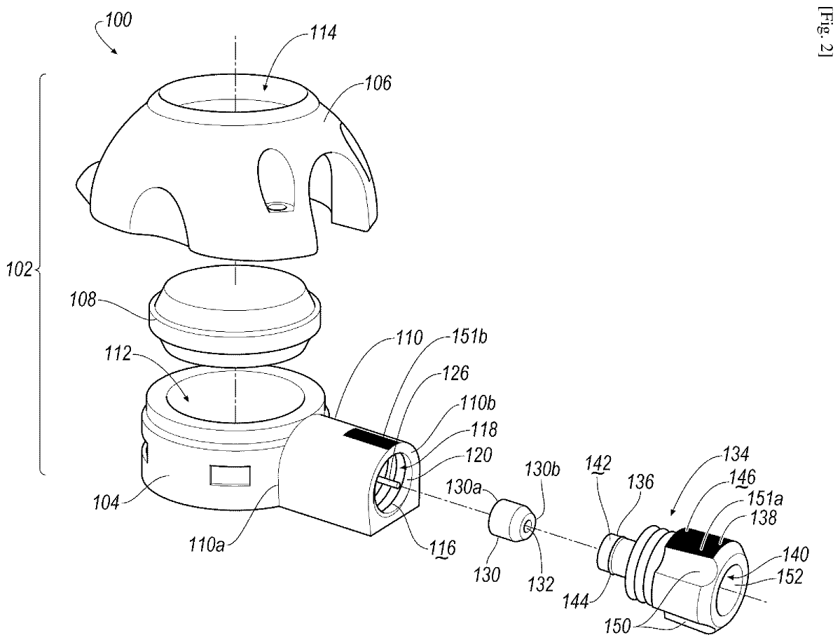

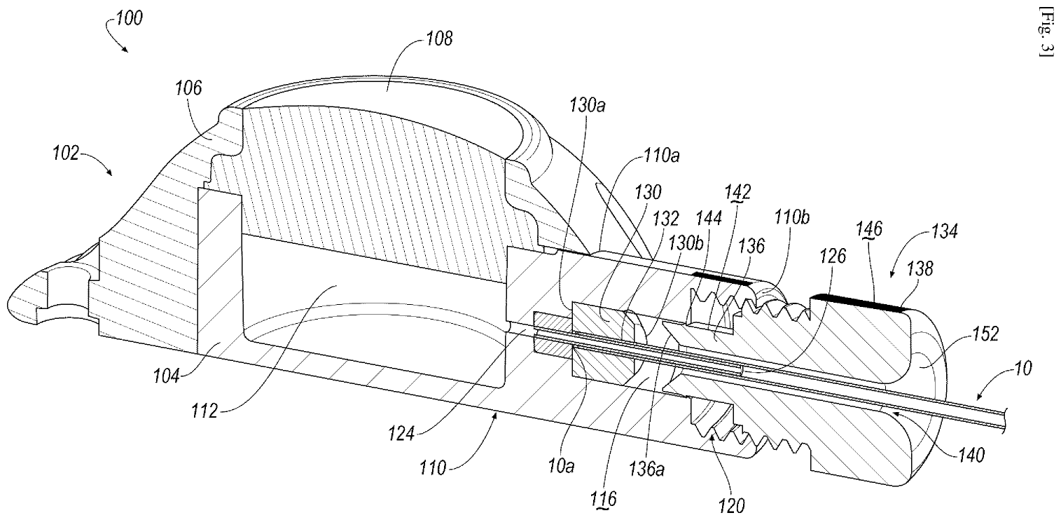

Referring to FIGS. 1-5B, a first example of a subcutaneous port assembly 100 is generally shown. The subcutaneous port assembly 100 includes a port 102 including a base 104, a cap 106, a septum 108, and a connector 110. The subcutaneous port assembly 100 may be surgically installed beneath the skin of a patient. As can be seen in FIGS. 2 and 3, the base 104 may define a reservoir 112, and the cap 106 may define an access aperture 114 that receives the septum 108, which may be a membrane formed of silicone or any other suitable material. As set forth in more detail below, the connector 110 may receive a distal end 10a of a catheter 10, and an opposite end of the catheter 10 may be connected to a vein, muscle, bone, tissue, or any other suitable anatomical structure or system of the patient. The catheter 10 may be a braided catheter having any suitable elasticity, strength, and rigidity. In some implementations, the catheter 10 has minimal elasticity or stretch along its longitudinal axis.

A healthcare provider, such as a physician, nurse, etc., may inject, via a syringe and needle, medication through the skin of the patient and through the septum to deliver the medication to the reservoir 112. The medication may then be delivered through the catheter 10 to the desired location for medication application. The base 104, the cap 106, and the connector 110 may be formed of any suitable material, such as titanium, stainless steel, cobalt-chrome alloy, nickel-titanium allow, gold, platinum, silver, iridium, tantalum, tungsten, polycarbonate, or any combination of the foregoing.

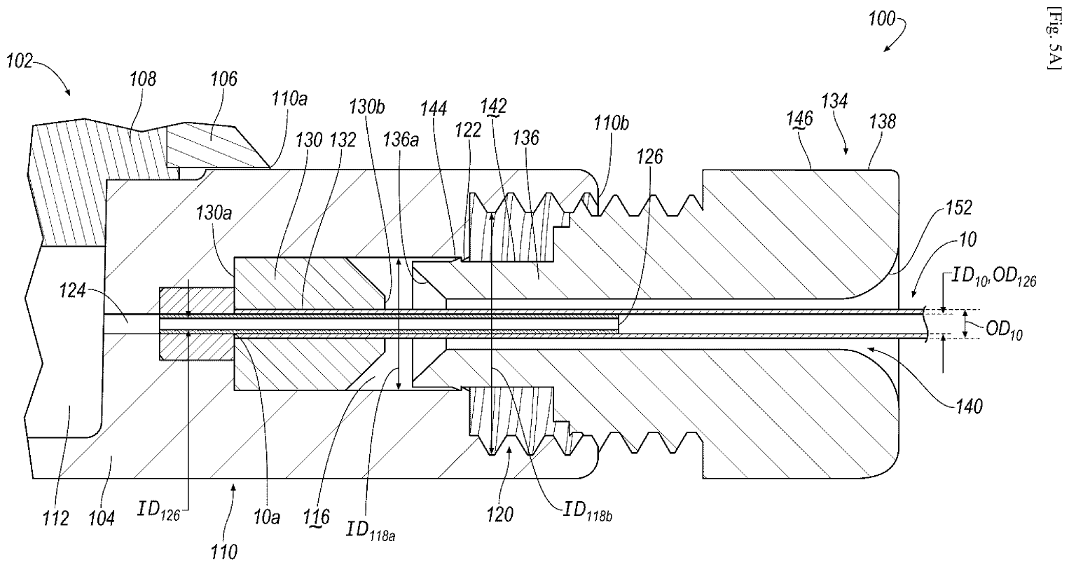

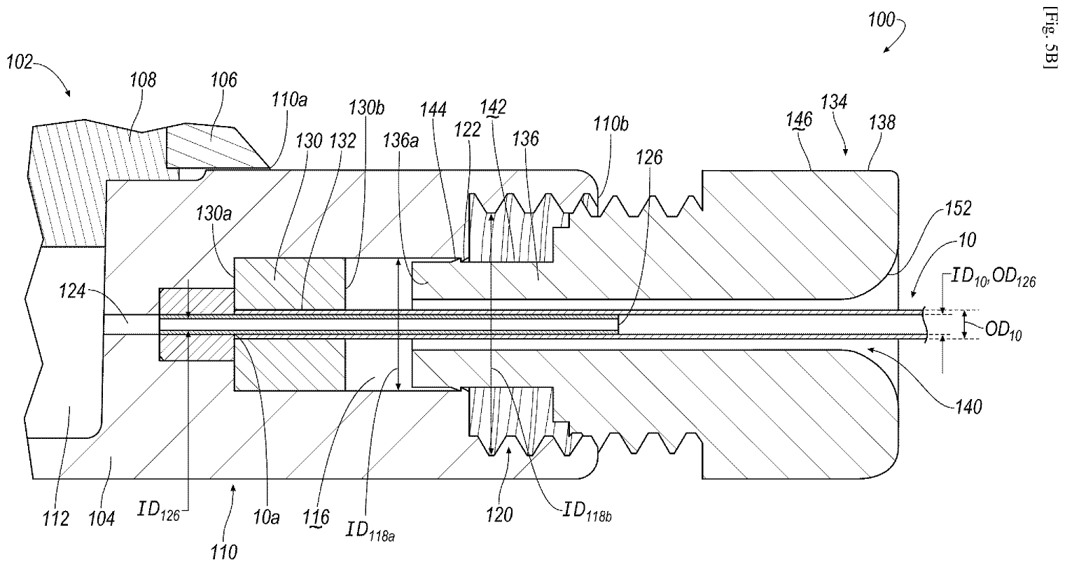

The connector 110 extends from a first end 110a attached to the base 104 to a distal end 110b spaced from the base 104. The connector 110 includes an inner surface 116 defining a socket 118. As indicated in FIG. 5A, the socket 118 includes a first inside diameter ID₁₁₈ₐ and a second inside diameter ID_{118b} greater than the first inside diameter ID₁₁₈ₐ. The first inside diameter ID₁₁₈ₐ may be located closer to the first end 110a than the second inside diameter ID_{118b}. The socket 118 may include a plurality of first threads 120 on the inner surface 116 at the portion having the second inside diameter ID_{118b}, e.g., the portion extending from the distal end 110b.

Referring to FIGS. 3-5B, the socket 118 may include a first radial protrusion 122 extending from the inner surface 116 at the portion having the first inside diameter ID₁₁₈ₐ. The first radial protrusion 122 may be a barb and have an angled portion that ramps up in a direction extending from the distal end 110b toward the first end 110a, and the first radial protrusion 122 may have a generally flat portion adjacent the angled portion and facing the first end 110a. The flat portion of the first radial protrusion 122 may be perpendicular to the inner surface 116 or may be undercut to provide an obstruction along the inner surface 116. In some implementations, the first radial protrusion 122 may be disposed at the end of the portion of the socket 118 having the first inside diameter ID₁₁₈ₐ. In other implementations, the first radial protrusion 122 may be disposed at any location of the socket 118 having the first inside diameter ID₁₁₈ₐ. The socket 118 may be in fluid communication with the reservoir 112 via a conduit 124 defined by one of the base 104 or the connector 110.

The port 102 includes a stem 126 extending from the base 104 and into the socket 118. The stem 126 includes an outer surface 128 having an outside diameter OD₁₂₆ that is less than the first inside diameter ID₁₁₈ₐ and the second inside diameter ID_{118b} of the socket 118. In some implementations, the outside diameter OD₁₂₆ of the stem 126 may be between approximately 0.2 mm and 1.0 mm. In some implementations, the outside diameter OD₁₂₆ of the stem 126 may be between approximately 0.3 mm and 0.7 mm. The stem 126 may extend along substantially an entire length of the socket 118, i.e., from the base 104 to the distal end 110b of the connector 110. In some implementations, the stem 126 may extend past or terminate before the distal end 110b of the connector 110. The stem 126 may be formed of any suitable type of material, such as titanium, stainless steel, cobalt-chrome alloy, nickel-titanium allow, gold, platinum, silver, iridium, tantalum, tungsten, polycarbonate, or any combination of the foregoing.

With continued reference to FIGS. 3-5B, the subcutaneous port assembly 100 includes a sleeve or sealing element 130 disposed within the socket 118 between the outer surface 128 of the stem 126 and the inner surface 116 of the socket 118. The sealing element 130 extends from a proximal end 130a to a distal end 130b. The proximal end 130a may be disposed at or near the end of the portion of the socket 118 having the first inside diameter ID₁₁₈ₐ. In some implementations, the distal end 130b may taper toward the longitudinal center of the sealing element 130, as shown in FIG. 5A. In other implementations, the distal end 130b may be generally flat or not tapered, as shown in FIG. 5B. The sealing element 130 may include a central bore 132 extending entirely through the sealing element 130, such that the sealing element 130 completely surrounds the stem 126. The sealing element 130 may be spaced apart from the outer surface 128 of the stem 126 when in an uncompressed state. Particularly, the surface defining the central bore 132 of the sealing element 130 is spaced apart from the outer surface 128 of the stem 126 by a distance greater than a wall thickness of the catheter 10. Accordingly, when the sealing element 130 is in the uncompressed state, the catheter can be inserted through the space between outer surface 128 of the stem 126 and the sealing element 130. The sealing element 130 may be formed of any suitable material, such as a foam, rubber, neoprene, silicone, etc.

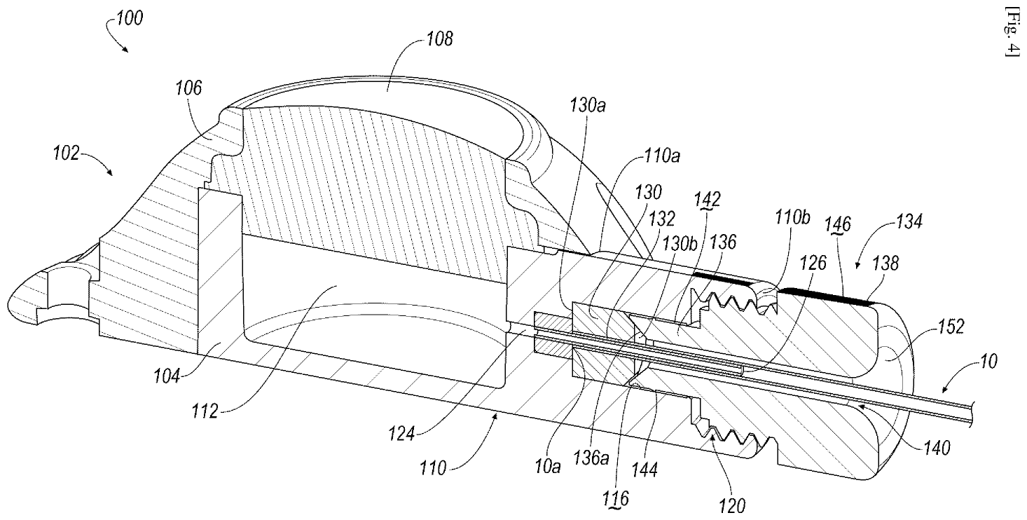

The subcutaneous port assembly 100 includes a locking member 134 having a plunger 136, a cap 138 adjacent the plunger 136, and a central bore 140 extending through the plunger 136 and the cap 138. The plunger 136 is received within the socket 118 from the distal end 110b. The plunger 136 includes a terminal end 136a facing the distal end 130b of the sealing element 130. In some implementations, the terminal end 136a may taper away from a longitudinal center of the plunger 136 to mate with the taper of the distal end 130b of the sealing element 130, as shown in FIG. 5A. In other implementations, the terminal end 136a of the plunger 136 may be substantially flat to abut the flat end of the distal end 130b of the sealing element 130, as shown in FIG. 5B. The plunger 136 is axially movable between a first position (FIG. 3) and a second position (FIG. 4) to selectively compress the sealing element 130 within the socket 118. The locking member 134 may be formed of any suitable material, such as titanium, stainless steel, cobalt-chrome alloy, nickel-titanium allow, gold, platinum, silver, iridium, tantalum, tungsten, polycarbonate, a polymeric material, a plastic material, or any combination of the foregoing.

The plunger 136 includes an outer surface 142 that extends into the first inside diameter ID₁₁₈ₐ of the socket 118. The outer surface 142 includes a second radial protrusion 144 extending from the outer surface 142. The second radial protrusion 144 may have an angled portion that ramps up in a direction extending from the terminal end 136a toward the cap 138, and the second radial protrusion 144 may have a generally flat portion adjacent the angled portion and facing the cap 138. The first radial protrusion 122 of the socket 118 and the second radial protrusion 144 of the plunger 136 are configured to selectively engage one another to retain at least a portion of the plunger 136 within the socket 118, as can be seen in FIGS. 5A and 5B. The angled portions of the first and second radial protrusions 122, 144 permit the plunger 136 to enter the first inside diameter ID₁₁₈ₐ of the socket 118, but, after the second radial protrusion 144 passes the first radial protrusion 122, the flat portions of the first and second radial protrusions 122, 144 restrict the plunger 136 from exiting the first inside diameter ID₁₁₈ₐ of the socket 118. Such a configuration ensures that, after proper installation, the locking member 134 does not disconnect from the port 102. While the first radial protrusion 122 and the second radial protrusion 144 are shown and described as having a shape resembling a right-angled triangle, it should be understood that any suitable shape is contemplated.

The cap 138 includes an outer surface 146 having a plurality of second threads 148 and a plurality of gripping members 150. The plurality of second threads 148 extend into the second inside diameter ID_{118b} of the socket 118 and are configured to engage with the plurality of first threads 120. The plunger 136 moves between the first position (FIG. 3) and the second position (FIG. 4) via the first threads 120 engaging with the second threads 148. The second threads 148 are configured such that a single rotation of the locking member 134 moves the plunger 136 between the first position (FIG. 3) and the second position (FIG. 4) to selectively compress the sealing element 130 within the socket 118 and secure the catheter. More specifically, when the plunger is in the second position (i.e., fully retracted) shown in FIG. 3, a single rotation of the locking member 134 in a clockwise direction moves the plunger 136 to the first position shown in FIG. 4 to compress the sealing element 130 to provide a locking force upon the catheter 10 in compliance with ISO 10555. Conversely, a full rotation in the counter-clockwise direction returns the plunger 136 from the fully-locked first position to the fully-unlocked second position shown in FIG. 3 to allow the catheter 10 to be easily disconnected from the stem 126.

The gripping members 150 may be a series of recesses, protrusions, or areas having a higher coefficient of friction to facilitate grasping and twisting of the cap 138 by, for example, a healthcare provider. The cap 138 may include a position indicator 151a configured to communicate a position of the locking member 134 to the user. Specifically, as discussed above, the first and second threads 120, 148 are configured such that a single rotation of the locking member 134 moves the locking member 134 between the fully-unlocked first position (FIG. 3) and the fully-locked second position (FIG. 4). Thus, the position indicator 151a communicates the rotational position of the cap 138 to the user such that the user can determine whether the locking member 134 is in the first position, the second position, or in an intermediate position between the first position and the second position. The position indicator 151a further cooperates with the first and second radial protrusions 122, 144 to prevent the locking member 134 from being inadvertently removed from the connector 110. For example, the position indicator 151a provides visual feedback to the user that that the locking member 134 is adjacent to or at the first position (i.e., fully retracted) while the first and second radial protrusions 122, 144 provide physical or tactile feedback to the user when the second radial protrusion 144 of the plunger 136 abuts the first radial protrusion 122 of the socket 118 at the first position. The redundant feedback (i.e. visual and tactile) advantageously minimizes the likelihood of the locking member 134 being removed from the socket 118, which is particularly advantageous when implemented with a catheter that is inserted within a patient.

In the illustrated example, the position indicator 151a is provided as a colored portion of the outer surface 146 corresponding to one of the protrusions of the gripping members 150. Optionally, a reference indicator 151b may be formed on the connector 110 to indicate a relative position of the locking member 134 relative to the connector 110. Each of the indicators 151a, 151b may be applied using a tampography process (i.e., pad printing). However, other coating processes (e.g., anodizing) may be used to form each of the indicators 151a, 151b. Additionally or alternatively, each of the indicators 151a, 151b may include tactile features formed in the outer surface 146, such as knurling or alignment slots.

The cap 138 includes a parabolic recess 152 extending toward the central bore 140. Upon installation of the catheter 10, the parabolic recess 152 is configured to direct the catheter 10 toward the central bore 140. For example, the distal end 10a of the catheter 10 may slide along the surface of the parabolic recess 152 toward the central bore 140.

In operation, the locking member 134 starts in the first position (FIG. 3) and the distal end 10a of the catheter 10 is inserted into the central bore 140 of the locking member 134. The distal end 10a of the catheter 10 continues along the central bore 140 in the socket 118, sliding over the outer surface 128 of the stem 126, and through the central bore 132 of the sealing element 130, until the distal end 10a of the catheter 10 terminates at the end of the socket 118. At this point, the catheter 10 surrounds the stem 126, but the catheter 10 may not be adequately secured to the stem 126. The locking member 134 is rotated via interaction with the cap 138, which causes the plunger 136 to move closer to the sealing element 130 as the first threads 120 engage with the second threads 148. The plunger 136 moves further into the socket 118 until the terminal end 136a of the plunger 136 engages the distal end 130b of the sealing element 130. The locking member 134 continues to rotate and move toward the second position (FIG. 4), causing the terminal end 136a of the plunger 136 to axially and radially compress the distal end 130b of the sealing element 130 toward the outside diameter OD₁₂₆ of the outer surface 128 of the stem 126, thus, securing the catheter 10 to the stem 126. In other words, as the locking member 134 moves from the first position to the second position, the sealing element 130 moves from an uncompressed state (i.e., spaced apart from the catheter) to a compressed state (i.e., contacting the catheter) to secure the catheter 10 to the stem 126.

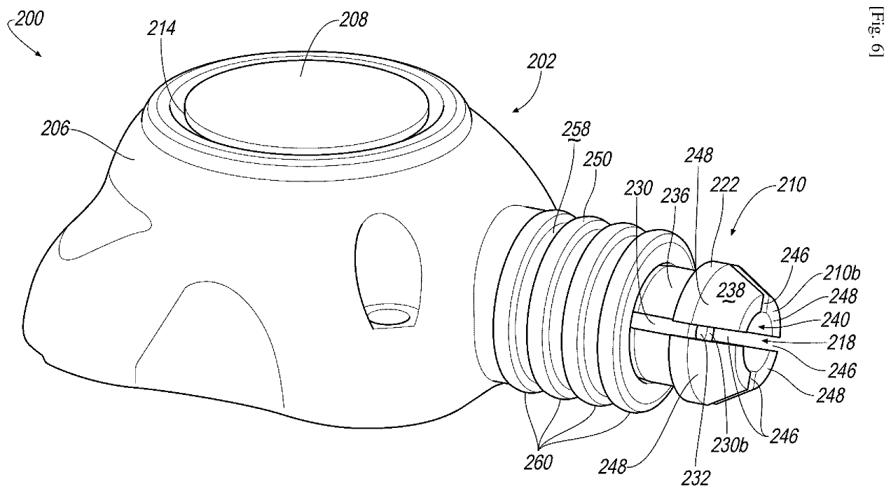

Referring to FIGS. 6-10, a second example of a subcutaneous port assembly 200 is generally shown. The subcutaneous port assembly 200 includes a port 202 having a base 204, a cap 206, a septum 208, and a connector 210. The base 204 defining a reservoir 212, the cap 206 defining an access aperture 214, and the septum 208 are substantially similar in structure and functionality to the base 104, the cap 106, and the septum 108 of the subcutaneous port assembly 100 as described above, and, as such, these components will not be described in detail.

The connector 210 extends from a first end 210a adjacent to the base 204 to a distal second end 210b spaced from the base 204. The connector 210 includes an inner surface 216 defining a socket 218. The socket 218 includes an inside diameter ID₂₁₈. The socket 218 includes at least one radial protrusion 220 extending from the inner surface 216 toward a center of the socket 218. In some implementations, the at least one radial protrusion 220 may include two radial protrusions 220, or any other suitable number of radial protrusions. The radial protrusions 220 may have a generally triangular shape, such as an equilateral triangle, or any other suitable shape. The socket 218 may be in fluid communication with the reservoir 212 via a conduit 224 defined by one of the base 204 or the connector 210.

The connector 210 includes a head portion 222, a neck portion 234, and a shoulder portion 236 between the head portion 222 and the neck portion 234. The neck portion 234 includes a first width W₂₃₄ at the first end 210a of the connector, e.g., at the portion connecting to the base 204, and the shoulder portion 236 includes a second width W₂₃₆ greater than the first width W₂₃₄ closer to the second end 210b of the connector 210. The socket 118 extends through the head portion 222, through the shoulder portion 236, and at least partially into the neck portion 234. The shoulder portion 236 includes a lip or first catch 244 on an outer surface of the shoulder portion 236. The first catch 244 may face toward the second end 210b of the connector 210, e.g., the first catch 244 may face toward the head portion 222.

The head portion 222 may include a tapered outer surface 238 that extends to the second end 210b of the connector 210. The head portion 222 includes a tapered recess 240 extending toward the socket 218. Upon installation of the catheter 10, the tapered recess 240 is configured to direct the catheter 10 toward the socket 218. For example, the distal end 10a of the catheter 10 may slide along the surface of the tapered recess 240 toward the socket 218.

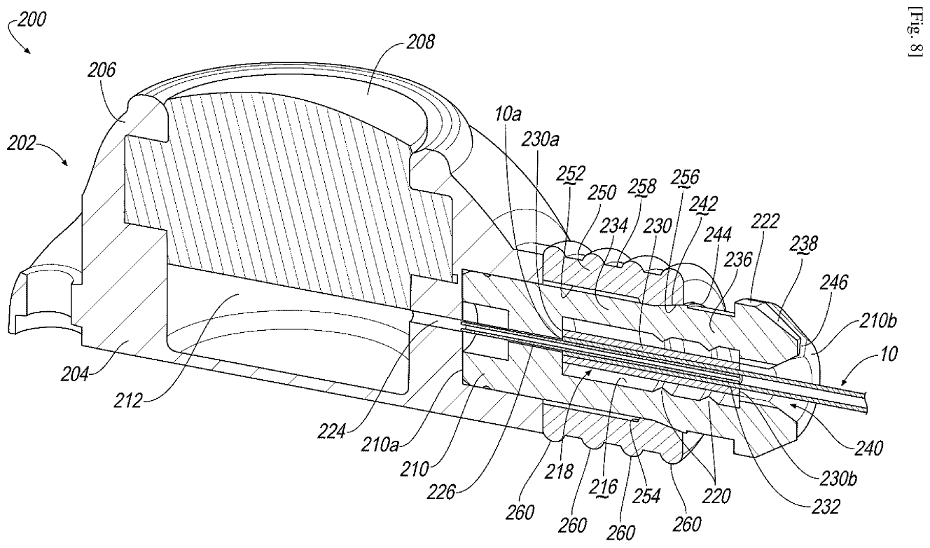

Referring to FIGS. 8-10, the connector 210 includes an outer biasing surface 242 extending from the outer surface of the neck portion 234 to the outer surface of the shoulder portion 236. The outer biasing surface 242 may be tapered relative to the neck portion 234 and the shoulder portion 236. For example, because the first width W₂₃₄ of the neck portion 234 is less than the second width W₂₃₆ of the shoulder portion 236, the outer biasing surface 242 increases in width from the neck portion 234 to the shoulder portion 236.

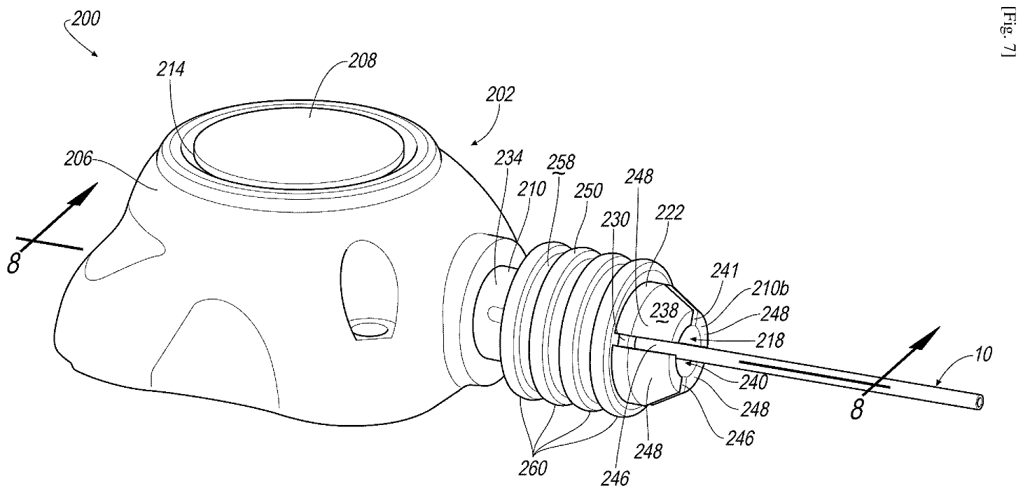

Referring to FIGS. 6 and 7, the connector 210 includes at least one slot 246 extending from the socket 218 through the head portion 222, the shoulder portion 236, and at least a portion of the neck portion 234. The at least one slot 246 may include four slots, or any other suitable number of slots. The slots 246 may cooperate to define at least one flexible tab 248 of the connector 210. For example, in implementations where there are four slots 246, there may be four flexible tabs 248 defined by the four slots 246. Each tab 248 may be spaced apart from an adjacent tab 248 by one of the slots 246.

The port 202 includes a stem 226 extending from the base 204 and into the socket 218. The stem 226 includes an outer surface 228 having an outside diameter OD₂₂₆ that is spaced inwardly from the inner surface 216 of the socket 218 and that is less than the inside diameter ID₂₁₈ of the socket 218. In some implementations, the outside diameter OD₂₂₆ of the stem 226 may be between approximately 0.2 mm and 1.0 mm. In some implementations, the outside diameter OD₂₂₆ of the stem 226 may be between approximately 0.3 mm and 0.7 mm. The stem 226 may be surrounded by the connector 210 and may extend along substantially an entire length of the socket 218, i.e., from the base 204 to the second end 210b of the connector 210. In some implementations, the stem 226 may extend past or terminate before the second end 210b of the connector 210, e.g., the stem 226 may terminate at or near the start of the head portion 222. The stem 226 may be formed of any suitable type of material, such as titanium, stainless steel, cobalt-chrome alloy, nickel-titanium allow, gold, platinum, silver, iridium, tantalum, tungsten, polycarbonate, or any combination of the foregoing.

Referring to FIGS. 8-10, the subcutaneous port assembly 200 includes a sleeve or sealing element 230 disposed within the socket 218 between the stem 226 and the inner surface 216 of the socket 218. The sealing element 230 extends from a proximal end 230a to a distal end 230b. The proximal end 230a may be disposed at or near the end of the portion of the socket 218 closer to the base 204. The sealing element 230 may include a central bore 232 extending entirely through the sealing element 230, such that the sealing element 230 completely surrounds the stem 226. The sealing element 230 may be spaced apart from the outer surface 228 of the stem 226 when in an uncompressed state. Particularly, the surface defining the central bore 232 of the sealing element 230 is spaced apart from the outer surface 228 of the stem 226 by a distance greater than a wall thickness of the catheter 10. Accordingly, when the sealing element 230 is in the uncompressed state, the catheter 10 can be inserted through the space between outer surface 228 of the stem 226 and the sealing element 230. The sealing element 230 may be formed of any suitable material, such as a foam, rubber, neoprene, silicone, etc.

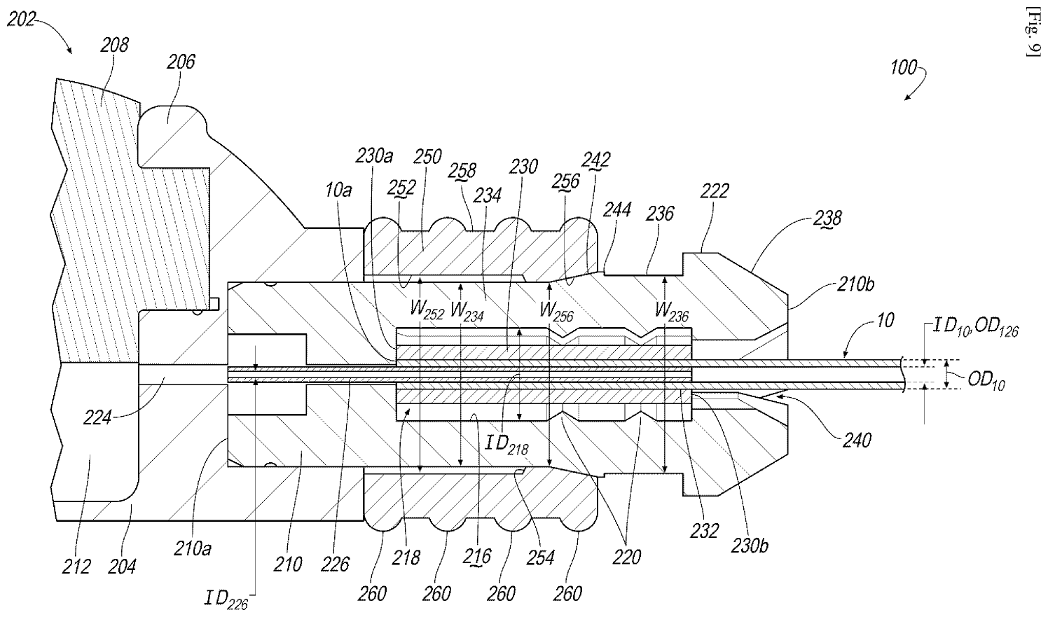

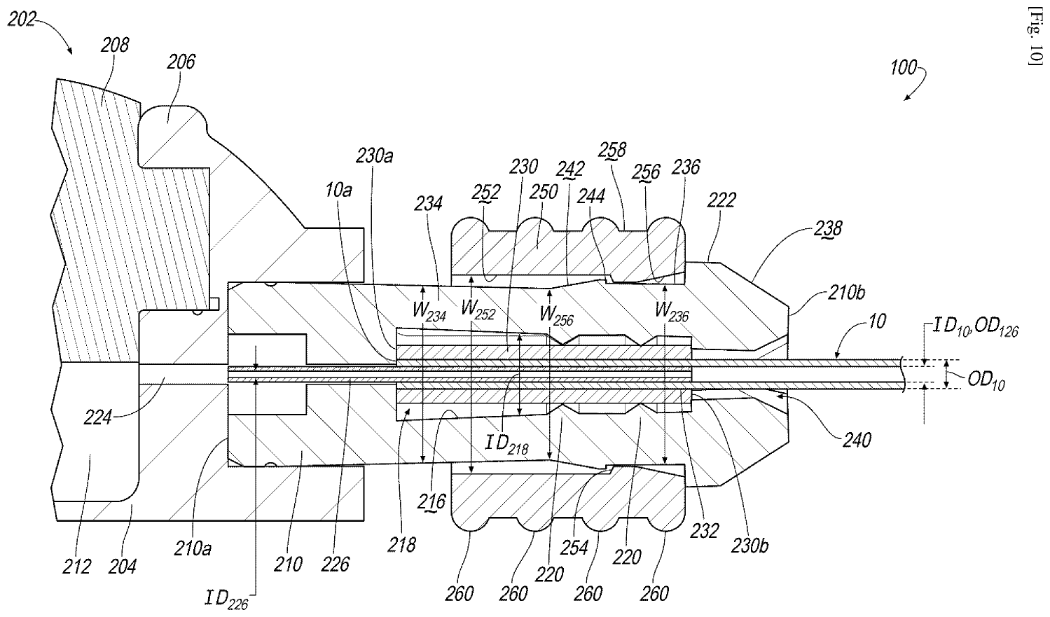

The subcutaneous port assembly 200 includes a collar 250 that is slidable relative to the connector 210 between a first, unlocked position (FIGS. 6 and 9) and a second, locked position (FIGS. 7 and 10). The collar 250 includes a first inner surface 252 having a third width W₂₅₂ that is greater than the first width W₂₃₄ of the neck portion 234 of the connector 210. The first inner surface 252 includes a lip or second catch 254 extending from the first inner surface 252. The second catch 254 is configured to engage with the first catch 244 extending from the shoulder portion 236. The catches 244, 254 are oriented to permit movement of the collar 250 away from the base 204, but, after the second catch 254 passes the first catch 244, the first catch 244 engages with the second catch 254 to restrict movement of the collar 250 toward the base 204. The collar 250 includes a second inner surface 256 having a fourth width W₂₅₆ that increases in a direction moving away from the base 204. That is, a portion of the second inner surface 256 is beveled to cooperate with the flared outer biasing surface 242 of the connector 210. The fourth width W₂₅₆ of the second inner surface 256 is greater than the first width W₂₃₄ of the neck portion 234 and less than the second width W₂₃₆ of the shoulder portion 236. In the unlocked position, the second inner surface 256 of the collar 250 is adjacent the neck portion 234. In the locked position, the second inner surface 256 of the collar 250 is adjacent the shoulder portion 236. The second catch 254 is disposed between the first inner surface 252 and the second inner surface 256.

The collar 250 includes an outer surface 258 including a plurality of gripping members 260. The gripping members 260 may be a series of recesses, protrusions, or areas having a higher coefficient of friction to facilitate grasping and sliding of the collar 250 by, for example, a healthcare provider. Additionally or alternatively, the inner surfaces 252, 256 of the collar 250 may include a plurality of first threads, and the outer surface of the connector 210 may include a plurality of corresponding second threads configured to engage the first threads, such that the collar 250 may rotate about the connector 210 to move from the unlocked position to the locked position. The collar 250 may be formed of any suitable material, such as titanium, stainless steel, cobalt-chrome alloy, nickel-titanium allow, gold, platinum, silver, iridium, tantalum, tungsten, polycarbonate, a polymeric material, a plastic material, or any combination of the foregoing.

In operation, the collar 250 starts in the unlocked position (FIG. 6). As discussed above, in the unlocked position the collar 250 is arranged so that the second inner surface 256 is aligned with the neck portion 234 of the connector 210. As such, the shoulder portion 236, and more particularly, the tabs 248 of the shoulder portion 236, are not compressed by the collar 250. As shown in FIG. 9, in the uncompressed state, the radial protrusions 220 formed on the inner surface 216 of the socket 218 are spaced radially outwardly from the sealing element 230 so that the inner bore of the sealing element 230 is spaced apart from the stem 226.

With the collar 250 in the unlocked state, the distal end 10a of the catheter 10 is inserted into the socket 218. The distal end 10a of the catheter 10 continues along the socket 218, sliding over the outer surface 228 of the stem 226, and through the central bore 232 of the sealing element 230, until the distal end 10a of the catheter 10 terminates at the end of the socket 218. At this point, the catheter 10 surrounds and is coupled to the stem 226, but the catheter 10 may not be adequately secured to the stem 226. The collar 250 is slid from the unlocked position toward the locked position, e.g., until the second catch 254 passes the first catch 244. At this point, the engagement of the catches 244, 254 restricts the collar 250 from sliding back toward the base 204. As the collar 250 slides toward the locked position, the connector 210 radially deflects toward the sealing element 230 via the slots 246 by the second inner surface 256 of the collar 250 sliding along the outer biasing surface 242 of the connector 210. The connector 210 radially deflecting toward the sealing element 230 causes the radial protrusions 220 to radially compress the sealing element 230, which compresses the catheter 10 and secures the catheter 10 to the stem 226.

It should be understood that in addition to the foregoing description, other implementations and/or embodiments are also contemplated. Additionally, any of the implementations or embodiments, including any features of each implementation, may be combined or interchanged as suitable.

In some implementations, the catheter may be at least partially secured to a stem via a sleeve or insert that wraps around the catheter and is tightened in any suitable manner, including via threads, ridges, ribs, protrusions, radial compression, etc.

In some implementations, a port assembly includes a connector extending from a base to a distal end. The connector includes an inner cavity extending from the distal end of the connector, where an inside diameter of the cavity tapers along a direction away from the distal end. Similar to the examples above, a stem extends from the base and through the cavity, towards the distal end of the connector. The port assembly further includes a locking member having a tube configured to be axially inserted into the cavity of the connector. The shaft may be configured as a hollow shaft or tube having a passage formed axially therethrough. A plurality of slots or notches are formed through a distal end of the shaft to provide the distal end shaft with a plurality of flexible tabs. When the distal end of the shaft is inserted into the cavity of the connector, the fingers engage the tapered inside diameter of the cavity and are biased radially inwardly towards the stem. In use, the catheter is threaded over the stem prior to the locking member being engaged with the tapered portion of the cavity. The locking member is then moved towards the locking position so that the distal end of the shaft engages the tapered portion of the cavity. Here, the fingers are biased radially inwards to compress the catheter against the stem. In some instances, the cavity may include slots or detents formed therein, and the locking member may include corresponding ribs or protrusions. When the locking member is fully engaged with the cavity, the ribs on the screw cap may create a snap-fit connection to the connector to secure the catheter to the connector and to indicate that the locking member is properly seated.

In some implementations, a port assembly includes a catheter lock having a plurality of rigid, angled tabs extending radially around a flexible segment that may have a cylindrical shape. The catheter lock may wrap around an outer surface of the catheter, securing the catheter lock to the catheter, e.g., by being threaded through a screw and a over a pin of the catheter lock. Similar to the examples above, the port base may define a cavity and a stem may extend from the base through the cavity. The port base may include a plurality of slots extending from the cavity and configured to receive the plurality of tabs. The catheter lock may be inserted into the cavity with the angle of the tabs causing the tabs to compress the flexible segment such that the tabs flex toward a center of the flexible segment. The catheter lock continues further into the cavity until the slots receive the tabs and resist movement of the catheter lock away from the port base via engagement of the tabs and the slots. In such implementations, the catheter lock may be similar to a Bayonet Neill-Concelman (BNC) connector with the port base acting as the BNC male and the catheter lock acting as the BNC female.

In some implementations, a port assembly includes a port base that may extend from a proximal end to a distal end. The distal end may radially flex, e.g., via at least one slot extending through the port base, the port base being formed of a flexible material, etc. The port base may define a cavity with a stem extending through the cavity. A catheter lock may be wrapped around the catheter and temporarily secured to the catheter, e.g., via friction. The catheter lock may have a cylindrical shape and a longitudinal divide extending along the catheter lock, such that the catheter lock may be opened along the longitudinal divide to remove the catheter lock from attachment to the catheter. The catheter lock, including the catheter, may extend into the cavity of the port base, causing the distal end to flex radially outwardly to allow the catheter to wrap around an outer surface of the stem. The catheter lock may be removed from the cavity, causing the distal end to flex radially inwardly to compress the catheter to the stem. The catheter lock may then be opened via the longitudinal divide and disconnected from the catheter.

In some implementations, a port assembly may define a cavity and a stem extending through the cavity. The port assembly may include a collet wrapped around the catheter, and the catheter may include a cover with a snap feature. The catheter may be threaded over the stem, through the collet, and the cover with the snap feature may secure the catheter to the collet. That is, the cover may wrap around the collet, which may wrap around the catheter, which may wrap around the stem, creating an interference fit between these components to secure the catheter to the stem. The collet may have barbs or flares to engage with portions of the cover, such as a slots, recesses, etc.

In some implementations, the catheter may be connected to a locking cap having a generally cylindrical shape and extending from a proximal end to a distal end including a silicone gasket attached to the distal end. The locking cap may include a keyed slot having a first portion that extends along a longitudinal direction of the cylindrical locking cap, a second portion extending perpendicular to the first direction around an outer periphery of the locking cap, and a third portion extending parallel to the first portion, e.g., the keyed slot may resemble a “J” shape. The catheter may be threaded over a stem extending through a cavity of the port base. The port base may include a pin extending from an outer surface of the stem. The locking cap may slide over the stem and the locking cap may be rotated to receive the pin within the keyed slot, i.e., the third portion of the keyed slot, thus securing the locking cap to the port base. In the locked position, the silicone gasket may abut the port base and be compressed to maintain the catheter in the locked position.

The terminology used herein is for the purpose of describing particular exemplary configurations only and is not intended to be limiting. As used herein, the singular articles “a,” “an,” and “the” may be intended to include the plural forms as well, unless the context clearly indicates otherwise. The terms “comprises,” “comprising,” “including,” and “having,” are inclusive and therefore specify the presence of features, steps, operations, elements, and/or components, but do not preclude the presence or addition of one or more other features, steps, operations, elements, components, and/or groups thereof. The method steps, processes, and operations described herein are not to be construed as necessarily requiring their performance in the particular order discussed or illustrated, unless specifically identified as an order of performance. Additional or alternative steps may be employed.

When an element or layer is referred to as being “on,” “engaged to,” “connected to,” “attached to,” or “coupled to” another element or layer, it may be directly on, engaged, connected, attached, or coupled to the other element or layer, or intervening elements or layers may be present. In contrast, when an element is referred to as being “directly on,” “directly engaged to,” “directly connected to,” “directly attached to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

The terms first, second, third, etc. may be used herein to describe various elements, components, regions, layers and/or sections. These elements, components, regions, layers and/or sections should not be limited by these terms. These terms may be only used to distinguish one element, component, region, layer or section from another region, layer or section. Terms such as “first,” “second,” and other numerical terms do not imply a sequence or order unless clearly indicated by the context. Thus, a first element, component, region, layer or section discussed herein could be termed a second element, component, region, layer or section without departing from the teachings of the example configurations

The foregoing description has been provided for purposes of illustration and description. It is not intended to be exhaustive or to limit the disclosure. Individual elements or features of a particular configuration are generally not limited to that particular configuration, but, where applicable, are interchangeable and can be used in a selected configuration, even if not specifically shown or described. The same may also be varied in many ways. Such variations are not to be regarded as a departure from the disclosure, and all such modifications are intended to be included within the scope of the disclosure.

1. A subcutaneous port assembly comprising:
a base;
a connector extending from a first end attached to the base to a distal end and including an inner surface defining a socket having an inside diameter;
a stem extending from the base and into the socket, the stem including an outer surface having an outside diameter that is less than the inside diameter of the socket;
a sealing element disposed within the socket between the stem and the inner surface of the socket; and
a locking member having a plunger received within the socket from the distal end and having a terminal end facing the sealing element, the plunger axially movable between a first position and a second position to selectively compress the sealing element within the socket.

2. The subcutaneous port assembly of claim 1, wherein the stem is surrounded by the sealing element.

3. The subcutaneous port assembly of any of claim 1 or 2, wherein the sealing element is spaced apart from the outside diameter of the outer surface of the stem by a first distance in an uncompressed state.

4. The subcutaneous port assembly of any of claims 1-3, wherein the plunger in the second position is configured to axially and radially compress the sealing element towards the outside diameter of the outer surface of the stem.

5. The subcutaneous port assembly of any of claims 1-4, wherein the locking member further includes a cap attached to the plunger, the cap including a plurality of first threads and the connector includes a plurality of second threads engaged with the first threads.

6. The subcutaneous port of claim 5, wherein the cap includes a position indicator.

7. The subcutaneous port of claim 6, wherein the position indicator is configured to communicate a rotational position of the cap.

8. The subcutaneous port of claim 6 or 7, wherein the cap includes a gripping member, the position indicator being provided on the gripping member.

9. The subcutaneous port assembly of any of claims 5-8, wherein the plunger moves between the first position and the second position via the first threads engaging with the second threads.

10. The subcutaneous port assembly of any of claims 1-9, wherein the socket includes a first radial protrusion on the inner surface and the plunger includes a second radial protrusion on an outer surface of the plunger, the first radial protrusion and the second radial protrusion configured to selectively engage one another to retain at least a portion of the plunger within the socket.

11. The subcutaneous port assembly of any of claims 1-10, wherein the sealing element includes a tapered end facing the terminal end of the plunger, the terminal end of the plunger configured to axially and radially compress the tapered end of the sealing element when the plunger moves from the first position to the second position.

12. A subcutaneous port assembly comprising:
a base;
a connector extending from a first end adjacent to the base to a second end, the connector including a neck portion having a first width at the first end and a shoulder portion having a second width closer to the second end, and a socket defined by an inner surface extending through the connector from the second end;
a stem extending from the base into the socket, the stem including an outer surface spaced inwardly from the inner surface of the socket;
a collar including an inner biasing surface having a third width that is greater than the first width of the neck portion and less than the second width of the shoulder portion, the collar operable between a first position where the inner biasing surface is adjacent to the neck portion and a second position where the inner biasing surface is adjacent to the shoulder portion; and
a sealing element disposed within the socket between the stem and the inner surface of the socket, the sealing element surrounding the stem when the collar is in the first position and compressed against the stem when the collar is in the second position.

13. The subcutaneous port assembly of claim 12, wherein the stem is surrounded by the connector.

14. The subcutaneous port assembly of any of claim 12 or 13, wherein the collar in the second position is configured to radially compress the sealing element to secure a catheter between the sealing element and the stem.

15. The subcutaneous port assembly of any of claim 12-14, wherein the connector includes at least one slot extending from the socket through the shoulder portion.

16. The subcutaneous port assembly of claim 15, wherein when the collar moves from the first position to the second position, the connector radially deflects toward the sealing element via the at least one slot.

17. The subcutaneous port assembly of any of claim 15 or 16, wherein the inner biasing surface is tapered and the connector includes a tapered outer biasing surface, and wherein when the collar moves from the first position to the second position, engagement of the inner biasing surface and the tapered outer surface causes the connector to radially deflect toward the sealing element and the sealing element to radially compress and secure a catheter to the stem.

18. The subcutaneous port assembly of claim 17, wherein the tapered outer surface of the connector is disposed between the neck portion and the shoulder portion.

19. The subcutaneous port assembly of any of claims 15-18, wherein the at least one slot includes a plurality of slots defining a plurality of flexible tabs of the connector, each flexible tab of the connector being spaced from an adjacent flexible tab of the connector by one of the slots.

20. A locking mechanism for a subcutaneous port assembly, the locking mechanism comprising:
a connector having a socket and a plurality of threads at a first end of the socket;
a stem disposed within the socket;
a sealing element disposed between the stem and the connector within the socket;
a braided catheter having an end coupled to the stem and surrounded by the sealing element; and
a locking member slidably coupled to the connector and moveable between a first position and a second position along the connector to selectively change the sealing element between a compressed state and an uncompressed state around the catheter.

21. The locking mechanism of claim 20, wherein the locking member includes a plunger received within the socket and a cap coupled to the connector by threads, the cap being movable between a locked position and an unlocked position relative to the connector via engagement of the threads.

22. The subcutaneous port of claim 21, wherein the cap includes a position indicator.

23. The subcutaneous port of claim 22, wherein the position indicator is configured to communicate a rotational position of the cap.

24. The subcutaneous port of claim 21 or 22, wherein the cap includes a gripping member, the position indicator being provided on the gripping member.

25. The locking mechanism of any of claims 21-24, wherein the connector includes a first radial protrusion on an inner surface and the locking member includes a second radial protrusion on an outer surface, the first radial protrusion and the second radial protrusion configured to engage one another to secure at least a portion of the plunger to the connector.

26. The locking mechanism of any of claims 21-25, wherein the sealing element includes a sleeve having a tapered first end and the plunger includes a tapered second end configured to engage the tapered first end of the sleeve, and wherein the tapered first end and the tapered second end engage when the locking member moves from the first position to the second position, causing the sleeve to axially and radially compress and secure the braided catheter to the stem.
